(19) United States
(12) Patent Application Publication
(10) Pub. No.: US 2009/0053174 A1
(43) Pub. Date: Feb. 26, 2009

(54) EJECTION LIQUID, EJECTION METHOD, METHOD FOR FORMING LIQUID DROPLETS, LIQUID EJECTION CARTRIDGE AND EJECTION APPARATUS

(75) Inventors: Hideki Kaneko, Yokohama-shi (JP); Masaru Sugita, Tokyo (JP); Yohei Masada, Kawasaki-shi (JP); Takeshi Miyazaki, Yokohama-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/909,249

(22) PCT Filed: Apr. 27, 2006
(86) PCT No.: PCT/JP2006/309215

§ 371 Date: Sep. 20, 2007

(30) Foreign Application Priority Data
May 2, 2005 (JP) 2005-133993

(51) Int. Cl. A61K 38/21; A61K 38/00; A61K 39/395; A61M 15/00; B41J 2/175

(52) U.S. Cl. 424/85.4; 514/2; 514/3; 424/130.1; 128/203.12; 347/86

(57) ABSTRACT

There are provided an ejection liquid that contains at least one of proteins and peptides and can be ejected stably by an ink jet system, and a method and apparatus for ejecting a liquid containing at least one of proteins and peptides using the ejection liquid. A specific amine is added to an aqueous solution of at least one of proteins and peptides to thereby improve the applicability to ejection by the ink jet system.

### TECHNICAL FIELD

The present invention relates to a liquid composition comprising at least one of proteins and peptides suitable for forming liquid droplets, a method for forming liquid droplets, and an ejection apparatus using the method.

### BACKGROUND ART

At present, many attempts are being made to utilize a protein solution as liquid droplets. Applications of forming liquid droplets technique of a protein solution include, for example, transmucosal administration as a drug delivery system and biochips and biosensors that require a very small amount of a protein. Further, also in control of protein crystals and screening of biologically active substances, methods of using fine protein liquid droplets are attracting attention (See Japanese Patent Application Laid-Open No. 2002-355025 and Allain L R et al. “Fresenius J. Anal. Chem.” 2001, Vol. 371, pp. 146-150, and also Howard, E I, Cachau R E “Biotechniques” 2002, Vol. 33, pp. 1302-1306).

In recent years, proteins, in particular useful proteins, such as enzymes and proteins having biological activity, can be mass-produced through the gene recombination technology, and liquid droplet formation of a protein can become a useful means for discovery, utilization and application of a protein as a new drug. Among others, a means for administering various drugs to patients using fine liquid droplets is becoming more important. Especially, the means is important for administration of not only proteins and peptides but other biological materials via the lung. In the lungs, since the surface area of the lung alveoli is as large as 50 m² to 140 m², and since the epithelium, which is an absorption barrier, is as thin as 0.1 μm, and further since the enzyme activity is lower as compared with that in the digestive tract, administration via the lung has been attracting attention as an administration route alternative to injection of a polymer-peptide drug represented by insulin.

In general, the intrapulmonary deposition of fine liquid droplets of a drug is known to be dependent upon their aerodynamic particle sizes. Among others, for delivery to the lung alveoli that is located deep inside of the lung, it is essential to develop an administration form and a stable formulation that can provide highly reproducible administration of liquid droplets having a narrow particle-size distribution of 1 μm to 5 μm.

Hitherto, there have been known several methods of administering a formulation into the body, in particular, to the respiratory organ or the periphery thereof, which are exemplified as follows.

In a metered dose inhaler (MDI) for a suspension aerosol form, a liquefied noncombustible or nonflammable gas is utilized as a propellant and a unit volume of the liquefied gas used for a single spraying is defined to attain the metered dose. However, there remain problems in controlling of the diameter of liquid droplets based on the unit volume of the liquefied gas, and it is difficult to say that the propellant is good for health.

Further, in atomization by a spray method of a liquid formulation using water or ethanol as a solvent, the liquid formulation is released through a capillary together with a pressurized carrier gas to be thereby converted into fine liquid droplets. There, in principle, the amount of atomization may be controlled by defining the amount of the liquid formulation supplied to the capillary flow path, but it is difficult to control the diameter of liquid droplets.

In particular, in atomization by the spray method, because the pressurized gas used in the process of converting the liquid formulation into fine liquid droplets is also used as a gas flow for carrying atomized fine liquid droplets, it is structurally difficult to change the amount of fine liquid droplets (density) floating in the carrier gas flow depending on the purpose.

As a method of producing liquid droplets with a narrow particle size distribution, there has been reported that a liquid droplet forming apparatus based on the principle of liquid ejection is used for ink jet printing is used to generate extremely fine liquid droplets and utilize them (see U.S. Pat. No. 5,894,841 and Japanese Patent Application Laid-Open No. 2002-248171). Here, in liquid ejection using this kind of an ink jet system, a liquid to be ejected is guided to a small chamber and a pressing force is applied to the liquid to eject liquid droplets through an orifice. Examples of such pressing methods include a method of using a electrothermal transducer such as a thin film resister to generate bubbles thereby ejecting liquid droplets through an orifice (ejection orifice) disposed on an upper part of the chamber (Thermal Ink Jet System), a method of using a piezoelectric vibrator to directly eject a liquid through an orifice disposed on an upper part of a chamber (Piezo Ink Jet System) and the like. The chamber into which the liquid is introduced and the orifice are integrated into a print head element, which is connected to a liquid supply source as well as to a controller that controls the ejection of liquid droplets.

To make a drug to be absorbed from the lung, accurate control of the administration amount is needed, especially in the case of a protein formulation, so that the liquid droplet formation based on the principle of the ink jet system, which allows the control of the ejection amount, is highly preferable. In addition, although sure ejection of a liquid is required, ejection of a protein solution having only surface tension and viscosity controlled is unstable, so that there have been cases where it is difficult to attain ejection with high reproducibility and efficiency.

A problem accompanying the liquid droplet formation of proteins or peptides based on the principle of the ink jet system is a fragile nature of the three dimensional structure of proteins, and there are cases where destruction of the structure may result in aggregation and degradation of proteins. The physical forces applied to liquid droplets when they are formed based on the principle of the ink jet system, such as a pressure, a shearing force, or a high surface energy which is characteristic of fine liquid droplets, make the structure of many proteins unstable (a heat is further applied when using the thermal ink jet system). Especially, when forming liquid droplets by utilizing the ink jet system, an ejection liquid is required to have not only long-term storage stability but also resistance and stability against the above described various loads. That is, because the physical actions described above are much greater than a shearing force and thermal energy applied by general stirring and heat treatment (for example, in the case of a thermal ink jet system, it is considered that a temperature of 300° C. and a pressure of 90 atm are applied instantaneously), and because a plurality of physical forces are applied simultaneously, the stability of a protein is more easy to be lowered than in a situation in which the protein is normally treated. Therefore, conventional protein stabilizing techniques have been sometimes insufficient. If this problem occurs, the protein will aggregate during liquid droplet formation to clog a nozzle (orifice), so that ejection of liquid droplets becomes difficult.

Further, because the size of 1 μm to 5 μm of liquid droplets, which are suitable for pulmonary inhalation, is very much smaller than about 16 μm, which is a typical diameter of liquid droplets generated by currently commercially available printers, a larger surface energy and shearing force are applied to the liquid droplets. Therefore, it is very difficult to eject a protein as fine liquid droplets which are suitable for pulmonary inhalation. When considering such liquid droplet diameters, as a liquid ejection apparatus for a protein solution, it is preferable to use an apparatus that is inexpensive to produce and based on the principle of the thermal ink jet system which allows nozzles to be disposed in a high density.

On the other hand, methods known to stabilize proteins, in which a surfactant, glycerol, various sugars, a water-soluble polymer such as polyethylene glycol, albumin, and the like are added, are almost or completely ineffective for improving the ejection performance in protein ejection based on the thermal ink jet system in most cases.

As liquid compositions for use in pulmonary inhalation of liquid droplets produced by using the thermal ink jet system, there have been known liquid compositions which contain compounds for controlling surface tension and humectants (see International Publication No. WO2002/094342 gazette). Here, a surfactant and a water-soluble polymer such as polyethylene glycol and the like are added to improve the stability of a protein in a solution formed into liquid droplets by modifying the surface tension, viscosity and moisturizing activity of the solution.

However, no description about ejection stability is given in the International Publication No. WO2002/094342 gazette, and according to the investigation of the present inventors, it has been found that the effect of the addition of a surfactant and a water-soluble polymer is insufficient when the concentrations of the protein and peptide are high and that the additives themselves may inhibit the ejection stability. Further, it has also been found that most of the surfactants have no effect, and that the ejection stability of a protein solution is not determined by its surface tension, viscosity and moisturizing action. In other words, the aforementioned method is not a general method for stabilizing the ejection when a peptide or protein is ejected by the thermal ink jet system.

As described above, examples of the methods for ejecting a liquid sample by converting it into fine liquid droplets include the known ink jet system. The ink jet system, in particular as to the amount of liquid ejected after being converted into liquid droplets, is characterized by exhibiting a high controllability even in a very small amount of a liquid droplet. The fine liquid droplet ejection method of the ink jet system is known to include the vibration system utilizing a piezoelectric element or the like and the thermal ink jet system utilizing a microheater element. The vibration system utilizing the piezoelectric element or the like has a limitation in the size reduction of the utilized piezoelectric element, so that the number of ejection orifices provided per unit area is limited. Also, as the number of ejection orifices provided per unit area is increased, the production cost therefore becomes higher steeply. On the other hand, in the thermal ink jet system, the size reduction of a utilized microheater element is relatively easy, and when compared with the vibration system utilizing the piezoelectric element or the like, the number of ejection orifices provided per unit area can be increased, and the production cost thereof can be made much lower.

When applying the thermal ink jet system, the physical properties of a liquid to be ejected need to be adjusted to suitably control the atomization state and amount of fine liquid droplets ejected from respective ejection orifices. That is, the liquid to be ejected is prepared by designing the liquid composition, such as the type and composition of solvents, the concentration of a solute and the like so that an objective amount of a fine liquid droplet can be obtained. Further, various technical developments are in progress in the ejection mechanism for liquid droplets that is based on the principle of the thermal ink jet system, and a new technology to an ejection mechanism/method, by which extremely fine liquid droplets of a liquid volume of an order of sub-picoliter or femto-liter can be obtained, has been developed (see Japanese Patent Application Laid-Open No. 2003-154655), while an ordinary ink jet head installed in a printer ejects liquid droplets of a liquid volume of about several picoliters. For example, it may be supposed that when somatic cells of several μm in size are selected as an object for applying a drug, it becomes necessary to utilize the extremely fine liquid droplets described as individual liquid droplets to be ejected.

### DISCLOSURE OF THE INVENTION

It is, therefore, an object of the present invention to provide an ejection liquid (liquid composition) for stably ejecting liquid droplets containing at least one of proteins and peptides based on a principle of an ink jet system utilizing a thermal energy, and an ejection method and apparatus suitable for ejecting the ejection liquid.

According to a first aspect of the present invention, there is provided an ejection liquid to be ejected from an ejection orifice utilizing a thermal energy for ejection comprising:

at least one of proteins and peptides;

at least one selected from amines represented by the formula (1):

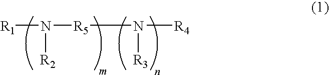

(wherein

R₁ and R₄ are each independently a hydrogen atom, a hydroxyl group, or a substituted or unsubstituted, linear or branched alkyl group having 1 to 8 carbon atoms:

each R₂ and each R₃ is independently a hydrogen atom, a hydroxyl group, or a substituted or unsubstituted, linear or branched alkyl group having 1 to 8 carbon atoms;

adjacent ones of R₁, R₂, R₃ and R₄ may be joined to form a substituted or unsubstituted heterocyclic ring;

each R₅ is independently an alkylene chain having 1 to 8 carbon atoms;

m is an integer of 0 or more; and

n is an integer of 1 or more) and salts thereof; and

a liquid medium comprising water as a main component.

According to a second aspect of the present invention, there is provided an ejection method comprising ejecting the aforementioned ejection liquid based on a principle of an ink jet system.

According to a third aspect of the present invention, there is provided a liquid ejection cartridge comprising a tank for containing the aforementioned ejection liquid and an ejection head.

According to a fourth aspect of the present invention, there is provided an ejection apparatus comprising the aforementioned cartridge, and a flow path and an orifice for leading a liquid ejected from a liquid ejecting portion of a head of the cartridge to an inhalation part of a user.

According to a fifth aspect of the present invention, there is provided a method of forming a droplet of a liquid comprising at least one of proteins and peptides by applying an energy for ejection to the liquid, which comprises the step of applying an energy for ejection to the liquid filled in a flow path to thereby eject a liquid droplet from an ejection orifice communicating with the flow path, wherein the liquid is the aforementioned ejection liquid.

According to the present invention, by adding the amine represented by the formula (1) or a salt thereof to a solution containing at least one of proteins or peptides, an ejection liquid can be obtained which can be ejected stably by application of a thermal energy. Moreover, by further adding a surfactant to the ejection liquid, a synergetic effect on ejection stability is obtained and it is possible to eject a protein solution of a much higher concentration. When the at least one of proteins and peptides has medicinal properties, by ejecting the ejection liquid by means of a portable ejection apparatus to form liquid droplets and by inhaling the liquid droplets, the at least one of proteins and peptides as medicinal properties can reach the lung and the medicinal properties can be absorbed. In addition, a substrate onto which the ejection liquid has been ejected according to the method described above may be utilized for production of biochips and biosensors, sensing, and screening of biomaterials.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a perspective view illustrating a method of ejecting a protein on a substrate;

FIG. 2 is a schematic view showing an example of a pattern for arranging a protein on a substrate;

FIG. 3 is a schematic view showing the internal structure of a head cartridge unit for an inhaler;

FIG. 4 is a perspective view showing an inhaler;

FIG. 5 is a perspective view showing a state in which an access cover of the inhaler of FIG. 4 is opened;

FIG. 6 is a graphical representation showing ejection amounts when an albumin solution is ejected by a thermal ink jet system; and

FIG. 7 is a model view of an experimental method performed in Example 25.

### BEST MODE FOR CARRYING OUT THE INVENTION

Preferred embodiments of the present invention will now be described in detail with reference to the accompanying drawings.

The term “protein” as herein employed refers to any polypeptide in which a number of amino acids are linked by peptide bonds and which is dissolved or dispersed in an aqueous solution.

Further, the term “peptide” as herein employed refers to a compound in which two or more amino acids are linked by peptide bond(s) and the number of amino acids is 100 or less.

Such proteins and peptides may be either chemically synthesized or purified from natural sources with natural proteins and recombinant peptides being typically used. Generally, in order to improve the efficacy of proteins and peptides, they may be chemically modified through covalent bonding of amino acid residues to proteins and peptides to thereby prolong their therapeutic effects.

When carrying out the present invention, various proteins and peptides, which are desired to form liquid droplets, may be used. Most typically, the liquid droplet formation of proteins and peptides according to the present invention may be utilized suitably for delivering therapeutically useful proteins and peptides to the lung.

Examples of the proteins and peptides available in the present invention include various hematopoietic factors such as calcitonin, blood coagulation factors, cyclosporin, G-CSF, GM-CSF, SCF, EPO, GM-MSF, CSF-1 and the like, cytokines including interleukins such as IL-1, IL-2, IL-3, IL-4, IL-5, IL-6, IL-7, IL-8, IL-9, IL-10, IL-11, IL-12 and the like, IGFs, M-CSF, thymosin, TNF and LIF. Further, examples of other proteins having a therapeutic effect available in the present invention include vasoactive peptides, interferons (alpha, beta, gamma or common interferon), growth factors or hormones, for example, human growth hormones or growth hormones of other animals (such as bovine, porcine or chicken growth hormones), insulin, oxytocin, angiotensin, methionine enkephalin, Substance P, ET-1, FGF, KGF, EGF, IGF, PDGF, LHRH, GHRH, FSH, DDAVP, PTH, vasopressin, glucagon, somatostatin and the like. Protease inhibitors, for example, leupeptin, pepstatin and metalloproteinase inhibitors (such as TIMP-1, TIMP-2 or other proteinase inhibitors) are used. Nerve growth factors such as BDNF and NT3 are also used. Plasminogen activating factors such as tPA, urokinase and streptokinase are also used. Peptide moieties of a protein, which contain all or a part of the main structure of the parental protein and possess at least a part of the biological properties of the parental protein, are also used. Analogs, for example, substitution or deletion analogs, or modified amino acids such as peptide analogs, and substances described above modified with a water-soluble polymer such as PEG, PVA and the like are also used. The fact that the aforementioned proteins can be delivered to the lung is explicitly shown in Critical Reviews in Therapeutic Drug Carrier Systems, 12 (2&3) (1995).

Further, for applications to the production of biochips and biosensors and to the screening of proteins and peptides, in addition to the proteins and peptides described above, the following proteins may be used: various enzymes such as oxidase, reductase, transferase, hydrase, lyase, isomerase, synthase, epimerase, mutase, racemase and the like; various antibodies such as IgG, IgE and the like, and receptors, and antigens to these; proteins and peptides used for diagnosis such as allergens, chaperonin, avidin, biotin and the like; and substances described above that are modified by a reagent for immobilization.

As the proteins and peptides to be contained in the ejection liquid, those having a molecular weight within the range of 0.5 kDa to 150 kDa may be used. Further, the content of the at least one selected from proteins and peptides in the ejection liquid may be chosen depending on the object or usage, and is preferably selected from the range of 1 ng/mL to 200 mg/mL.

The present inventors have conducted extensive studies and found that a solution obtained by adding the amine represented by the formula (1) to a solution comprising at least one of proteins and peptides as an active ingredient is suitable for forming stable liquid droplets by application of a thermal energy.

Here, the compound represented by the formula (1) contains a unit represented by —NR₂-R₅— and a unit represented by —NR₃—. R₁ and R₄ in the formula (1) represent, independently of each other, a hydrogen atom, a hydroxyl group, a substituted or unsubstituted linear alkyl group having 1 to 8 carbon atoms, or a substituted or unsubstituted branched alkyl group having 1 to 8 carbon atoms. R₂ and R₃ in the formula (1) represent, independently of each other, a hydrogen atom, a hydroxyl group, a substituted or unsubstituted linear alkyl group having 1 to 8 carbon atoms, or a substituted or unsubstituted branched alkyl group having 1 to 8 carbon atoms. Adjacent ones of R₁, R₂, R₃, and R₄ may be joined to form a substituted or unsubstituted heterocyclic ring. R₅ in the formula (1) represents an alkylene chain having 1 to 8 carbon atoms. m in the formula (1) represents an integer of 0 or more. n in the formula (1) represents an integer of 1 or more.

Further, when m is 2 or more, that is, when the unit represented by —NR₂-R₅— is present in plurality, R₂ and R₅ in the respective units represent, independently of each other, the atom, groups and chains as defined above. Also, when n is 2 or more, that is when the unit represented by —NR₃— is present in plurality, R₃ in the respective units represent, independently of each other, the atom and groups as defined above.

Further, a salt of the compound of the formula (1) may also be used.

Particular examples of the amines represented by the formula (1) include ammonia, ethylamine, diethylamine, trimethylamine, hydroxylamine, ethanolamine, 2-amino-1-propanol, 2-methylaminoethanol, 3-pyrrolidinol, piperidine, piperazine, morpholine, ethylenediamine, putrescine, spermidine, spermine and the like.

The content of the at least one selected from the amines represented by the formula (1) and salts thereof in the ejection liquid is preferably 0.0001 wt. % to 20 wt. % and more preferably 0.001 wt. % to 1 wt. %.

The reason for the great contribution of the amine represented by the formula (1) to the ejection stability is considered to be as follows. The amine represented by the formula (1) binds to the surface of a protein to increase “apparent net charge” toward the positive and to suppress collision between proteins. By this action, it is possible to prevent degradation and aggregation of proteins and peptides resulting from an energy load at the time of ejection based on the principle of the thermal ink jet system and also to stabilize the ejection.

Incidentally, when salts of the compound represented by the formula (1) are a drug, a pharmaceutically acceptable salt is preferably used.

Further, the present inventors have found that the stability of ejection can be maintained by adding an amine represented by the formula (1) and a surfactant together, even if the concentrations of the additives are remarkably low. By adding 0.1 to 20 parts by weight of a surfactant relative to 1 part by weight of an amine represented by the formula (1), the addition amount of the amine represented by the formula (1) to a solution containing the same concentration of an active ingredient can be reduced to 1/10 to ½.

As for the effect of the surfactant, it is considered that unlike the amines represented by the formula (1), the surfactant stabilizes the ejection by an action of preventing degradation of proteins and peptides as active ingredients and by another action of re-dissolving aggregated proteins and peptides. It is also considered that combination of these two different actions provides a synergistic effect to remarkably improve the ejection stability. Because a surfactant alone cannot provide these actions sufficiently, aggregation of proteins and peptides cannot be completely prevented thereby failing to secure the ejection stability.

The term “surfactant” as herein employed refers to those compounds having both a polar part and a non-polar part in one molecule, in which these two parts, which reduce an interfacial tension between two inmiscible phases by molecular arrangement at the interface and are capable of forming micelles, are respectively positioned at localized regions distant from each other in the molecule.

The surfactant includes, but not limited to, sorbitan fatty acid esters such as sorbitan monocaprylate, sorbitan monolaurate, sorbitan monopalmitate and the like; glycerol fatty acid esters such as glycerol monocaprylate, glycerol monomyristate, glycerol monostearate and the like; polyglycerol fatty acid esters such as decaglyceryl monostearate, decaglyceryl distearate, decaglyceryl monolinoleate and the like; polyoxyethylene sorbitan fatty acid esters such as polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate and the like; polyoxyethylene sorbit fatty acid esters such as polyoxyethylene sorbit tetrastearate, polyoxyethylene sorbit tetraoleate and the like; polyoxyethylene glycerol fatty acid esters such as polyoxyethylene glyceryl monostearate and the like; polyethylene glycol fatty acid esters such as polyethylene glycol distearate and the like; polyoxyethylene alkyl ethers such as polyoxyethylene lauryl ether and the like; polyoxyethylene polyoxypropylene alkyl ethers such as polyoxyethylene polyoxypropyleneglycol ether, polyoxyethylene polyoxypropylene propyl ether, polyoxyethylene polyoxypropylene cetyl ether and the like; polyoxyethylene alkylphenyl ether such as polyoxyethylene nonylphenyl ether and the like; polyoxyethylene cured castor oil such as polyoxyethylene castor oil, polyoxyethylene cured castor oil (polyoxyethylene hydrogenated castor oil) and the like; polyoxyethylene beeswax derivatives such as polyoxyethylene sorbit beeswax and the like; polyoxyethylene lanolin derivatives such as polyoxyethylene lanolin and the like; polyoxyethylene fatty acid amide of HLB6-18 such as polyoxyethylene stearic acid amide and the like; anionic surfactants, for example, alkyl sulfates with an alkyl group having 8-18 carbon atoms, such as sodium cetyl sulfate, sodium lauryl sulfate, sodium oleyl sulfate and the like; polyoxyethylene alkyl ether sulfates in which the average mole number of added ethyleneoxide is 2-4 and an alkyl group has 8-18 carbon atoms, such as sodium polyoxyetylene lauryl sulfate and the like; alkylbenzene sulfonates in which an alkyl group has 8-18 carbon atoms, such as sodium laurylbenzene sulfonate and the like; alkyl sulfosuccinates, in which an alkyl group has 8-18 carbon atoms, such as sodium lauryl sulfosuccinate and the like; natural surfactants, such as lecithin, glycerophsopholipids; sphingophospholipids, such as sphingomyelin and the like; sucrose fatty acid esters of fatty acids having 8-18 carbon atoms and the like. These surfactants may be added singly or in combination to the ejection liquid (liquid composition) of the present invention.

The preferable surfactant is polyoxyethylene sorbitan fatty acid esters, and the especially preferable surfactants are polyoxyethylene (20) sorbitan monolaurate, polyoxyethylene (4) sorbitan monooleate, polyoxyethylene (20) sorbitan monopalmitate, polyoxyethylene (20) sorbitan monostearate, polyoxyethylene (20) sorbitan tristearate, polyoxyethylene (20) sorbitan monolaurate, polyoxyethylene (5) sorbitan monooleate, and polyoxyethylene (20) sorbitan trioleate, with polyoxyethylene (20) sorbitan monolaurate and polyoxyethylene (20) sorbitan monooleate being most preferred. Further, polyoxyethylene (20) sorbitan monolaurate and polyoxyethylene (20) sorbitan monooleate are especially suitable for pulmonary absorption.

The concentration of the surfactant added, which may be dependent on the kinds of co-existing proteins and the like, may be for example, in the case of insulin, within the range of 0.001 wt. % to 20 wt. %.

In the embodiments of the present invention, antibacterial agents, fungicides (bacteriocides), preservatives or the like may be added to remove the influence of microorganisms. These include, for example, quaternary ammonium salts such as benzalkonium chloride and benzatonium chloride, phenol derivatives such as phenol, cresol, anisole and the like, benzoic acids such as benzoic acid, paraoxybenzoate ester, and sorbic acid.

In the embodiments of the present invention, in order to improve the physical stability during storage of the ejection liquid, there may be added oils, glycerol, ethanol, urea, cellulose, polyethylene glycol and alginates, and in order to increase the chemical stability, ascorbic acid, citric acid, cyclodextrin, tocopherol or other antioxidants may be added.

Further, a buffering agent may be added to adjust the pH of the ejection liquid. For example, there may be used, not only ascorbic acid, citric acid, diluted hydrochloric acid and diluted sodium hydroxide and the like, but also buffer solutions such as sodium hydrogen phosphate, sodium dihydrogen phosphate, potassium hydrogen phosphate, potassium dihydrogen phosphate, PBS, HEPES, and Tris.

Moreover, there may further be added, as an isotonic agent for liquid, aminoethylsulfonic acid, potassium chloride, sodium chloride, glycerol, or sodium hydrogen carbonate.

When the ejection liquid of the present invention is used as an atomizing liquid, there may be added as a flavoring agent or taste masking agent, sugars such as glucose and sorbitol, sweeteners such as aspartame, menthol, and other various flavors. Also, not only hydrophilic substances but hydrophobic compounds and oil-like materials may be used.

Further, various additives suitable for the usage of the ejection liquid, for example, surface regulators, viscosity regulators, solvents, moisturizers may be added in an appropriate amount, as needed. Specifically, hydrophilic binders, hydrophobic binders, hydrophilic thickeners, hydrophobic thickeners, glycol derivatives, alcohols and electrolytes are examples of the available additives and may be used singly or in combination. Further, as the various substances described above to be used as additives, it is preferable to use those which are for medicinal use and included in a national pharmacopoeia or the like as subsidiary components that may be added in preparing therapeutic liquid formulations or those which are accepted to be utilized in foods and cosmetics.

The addition percentage of the various substances described above to be mixed as additives varies depending on the types of objective proteins and peptides, which is, in general, preferably within the range of 0.001 to 40% by weight, and more preferably within the range of 0.01 to 20% by weight. Further, the addition amount of the additives described above varies depending on the type, amount and combination thereof, but it is preferable from the viewpoint of ejection property that the ratio is 0.1 to 200 parts by weight of the additive relative to 1 part by weight of the aforementioned proteins and peptides.

In the case of using the ejection liquid of the present invention for producing biochips and biosensors and for screening for a protein, it is possible to use substantially the same system as that of ink jet printers commercially available presently.

On the other hand, it is preferable that the liquid ejection apparatus of the present invention comprises an ejection head which is based on the principle of the thermal ink jet and is capable of ejecting fine liquid droplets of the ejection liquid by the thermal ink jet system and that a number of ejection units which constitute the head are constructed so that they can be driven independently of each other. At that time, it is preferable to adopt a liquid ejection cartridge of an integrated configuration such that wires which connect electrical connection portions serving for connection of a plurality of control signals or the like required for independently drive respective ejection units and the respective ejection units are integrated; and there are further provided a tank for storing the ejection liquid and a liquid flow path which is a means for supplying the ejection liquid from the tank to the ejection head designed based on the thermal ink jet principle.

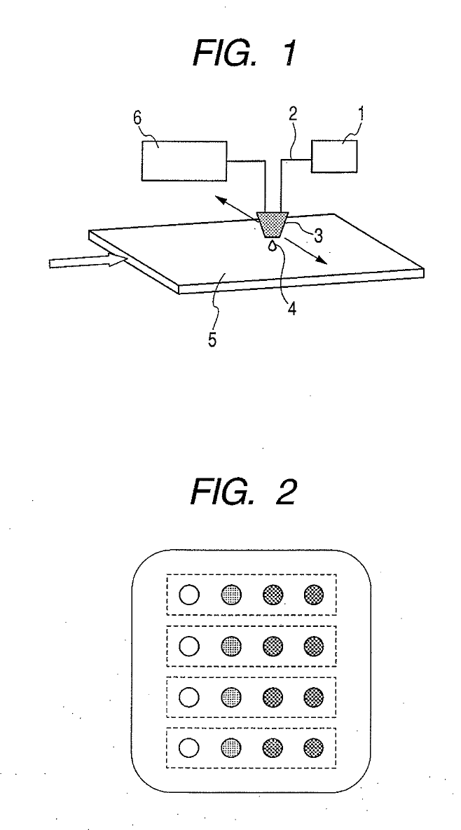

FIG. 1 is a schematic perspective view showing a apparatus for forming protein spots on a substrate using the ejection liquid according to the present invention. A substrate 5 is utilized as, for example, a detection plate on which fixed regions of standard substances such as proteins, peptides, enzymes, antibodies or the like for detect various substances contained in a sample are formed. A liquid ejection head 3 has at least a liquid path (not shown) in which an energy for ejection is applied to the liquid and an ejection orifice (not shown) which communicates with the liquid path. An energy for ejection is applied to the liquid which has been supplied to the liquid path from a tank 1 storing the liquid through a liquid supply path 2, and the liquid is ejected from the ejection orifice to a predetermined location on the surface of the substrate 5 in the form of a liquid droplet 4. The substrate 5 is disposed on a stage which allows positional adjustment in the directions parallel to the substrate surface indicated by the arrows, and by moving the stage, the arriving position of the liquid droplet 4 on the substrate 5 is adjusted. The timing of the ejection of the liquid droplet 4 is controlled by a controller 6 electrically connected to the ejection head 3. FIG. 2 is a plan view showing an example of an arrangement of protein spots on the surface of a substrate. In the example illustrated in the figure, a single kind of ejection liquid is used. However, by disposing in the ejection head a plurality of ejection units that eject different ejection liquids and that can be driven independently of each other, and by connecting a supply system of a predetermined ejection liquid to each unit, plural kinds of spots may be formed on the substrate. Further, by changing the amounts of liquid to be supplied to the respective spot forming sites, spots with different application amounts may be formed.

At that time, as the ejection head 3, there can be utilized ones of various types depending on the size and disposition density of spots formed on the substrate. When the volume of a single liquid droplet is in the order of subpicoliter or femtoliter, it is preferable to utilize the ejection head for ultrafine liquid droplets disclosed in Japanese Patent Application Laid-Open No. 2003-154655, which has a superior capability for controlling the liquid droplet volume in such order.

Next, description is made by taking as an example the case where the ejection liquid according to the present invention is used for atomization, in particular for an inhaler. As the inhaler, it is preferable to use an inhaler which has a part for converting an ejection liquid (liquid formulation) to fine liquid droplets and a part for incorporating the atomized fine liquid droplets into a carrier airflow, independently of each other. By taking the advantage of separating the atomizing part which converts the liquid into fine liquid droplets from the part in which the airflow containing the fine liquid droplets is formed, the amount of a protein and/or a peptide as effective components in the airflow, that is a predetermined dose per single administration, can be adjusted more uniformly when allowing an administration object to inhale the airflow. Also, by composing an ejection head in such a way that a plurality of ejection units each having a number of ejection orifices are provided so as to eject different effective components for every unit, the ejection amounts of a plurality of effective components can be controlled independently of each other.

Further, by utilizing an ejection head designed based on the thermal ink jet principle that allows disposition of ejection orifices at a high density per unit area as an atomizing mechanism, the size of an inhaler can be so reduced as to allow a user to bring it with him.

In the inhaler for pulmonary inhalation, it is important that the particle size distribution of liquid droplets contained in airflow is 1 μm to 5 μm and the range of particle size is narrow. Further, when it is utilized as a portable apparatus, the constitution of the apparatus needs to be compact.

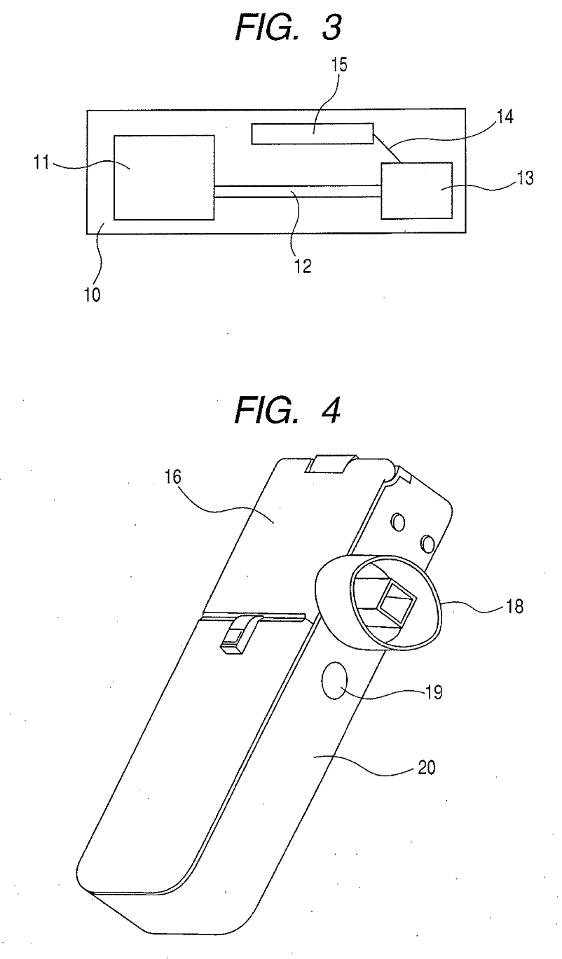

FIG. 3 is a schematic view showing the internal structure of an example of a liquid ejection part of such an inhaler. The liquid ejection part is composed as a head cartridge unit in which in a casing 10, a head portion 13, a tank 11 for storing an ejection liquid, a liquid path 12 for supplying the liquid from the tank 11 to the head portion 13, a controller 15 for driving the head portion 13, and a wire 14 for electrically connecting the head portion 13 and the controller 15 are formed integrally. The head cartridge unit is composed so as to be freely attachable to and detachable from the inhaler as needed. As the head portion 13, one having the constitution of the liquid droplet ejection head described in Japanese Patent Application Laid-Open No. 2003-154665 is suitably used.

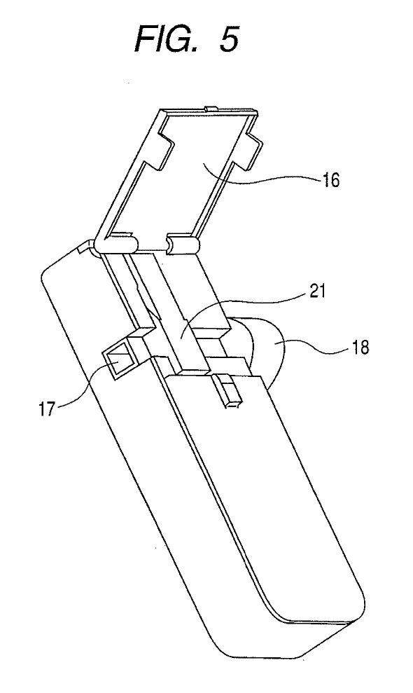

An example of a portable inhaler having a head cartridge unit composed in such a way will be described referring to FIGS. 4 and 5. The inhaler shown in FIGS. 4 and 5 has a constitution as an example which is designed to be compact such that a user can bring with him as a portable inhaler for used for a medical purpose.

FIG. 4 is a perspective view showing the appearance of the inhaler. In the inhaler, a housing is formed by an inhaler main body 20 and an access cover 16. In the housing, a controller, an electric source (battery) (not shown) and the like are housed. Reference numeral 19 denotes a power supply switch. FIG. 5 is a perspective view illustrating a state in which the access cover 16 is opened, and when the access cover 16 is opened, a connection portion between a head cartridge unit 21 and a mouthpiece 18 can be seen. Air is sucked into the inhaler from an air intake port 17 by the inhalation operation of a user and guided to enter the mouthpiece 18 and is then mixed with liquid droplets ejected from the ejection port provided in the head portion 13 (see FIG. 13) of the head cartridge unit 21 thereby forming a mixed airflow. The mixed air flow moves to a mouthpiece exit having such a shape that a person can put it in his mouth. By putting the tip of the mouthpiece into the mouth and holding it between the teeth and then breathing in, the user can inhale efficiently the droplets ejected from the liquid ejection part of the head cartridge unit.

Incidentally, the head cartridge unit 21 may be composed so as to be attachable to and detachable from the inhaler as needed.

By adopting the constitution such as shown in FIGS. 4 and 5, the fine liquid droplets formed can naturally be delivered into the throat and trachea of an administration object. Thus, the amount of atomized liquid (administration amount of effective component) is not dependent on the volume of breathed-in air but is controllable independently.

### EXAMPLES

### Reference Example 1

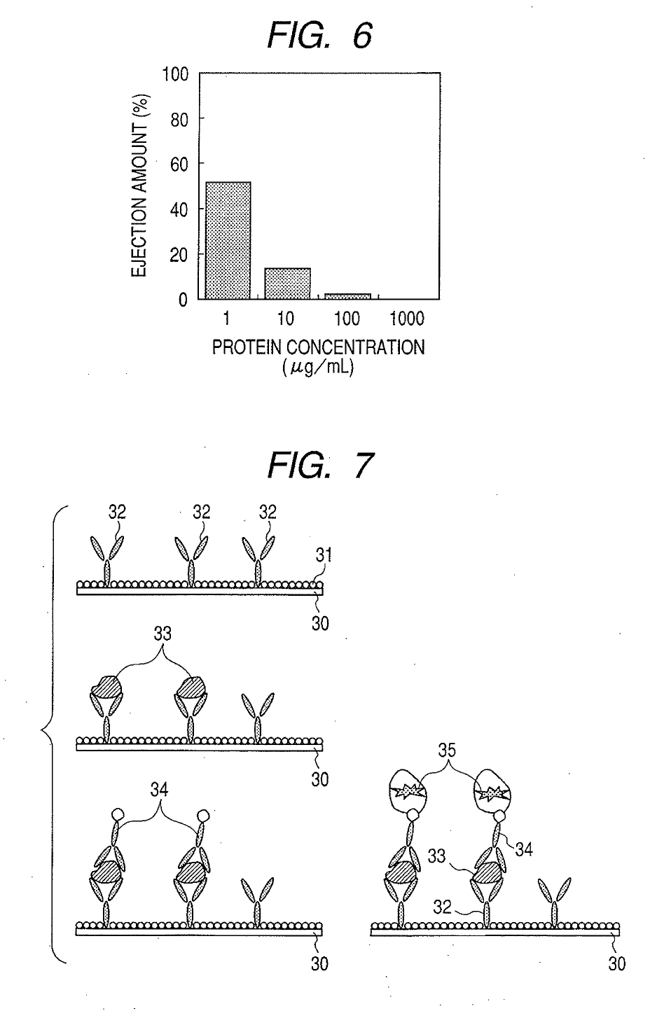

Before describing Examples, for better understanding of the difficulty of ejecting a protein solution, there are shown the ejection amounts when protein alone is ejected by the thermal ink jet system. Solutions of albumin in PBS at various concentrations were used as the protein solution and were ejected using a liquid ejection apparatus which was a thermal ink jet printer (PIXUS950i (trade name); manufactured by Canon Inc.) modified such that the solution could be recovered. The ejection amount of each albumin solution (volume of a single liquid droplet) was expressed in terms of percent with the ejection amount (volume of a single liquid droplet) when pure water was similarly ejected being defined as 100%. The results are shown in FIG. 6.

It can be seen from FIG. 6 that even at a low albumin concentration of 1 μg/mL, the ejection stability is not perfect, and as the protein concentration becomes higher, the ejection amount changes and gradually becomes zero. When the ejection amount changes greatly depending on a protein concentration, it may become necessary to adjust the ejection drive conditions for each protein concentration, for example, in quantitative disposition of protein spots on a substrate. Further, when utilized for a drug inhaler, it may become necessary to adjust the ejection drive conditions for each protein concentration to make the amounts of protein uniform for unit administrations. Moreover, in the inhaler, the liquid must be ejected as droplets of a further smaller diameter, and thus it is considered that the ejection of protein solution would be more difficult.

The present invention will be described below in more detail with reference to Examples, but these Examples are particular examples provided for deeper understandings, and the present invention is not limited by these particular examples. Here, “%” means % by weight.

### Examples 1-9 and Comparative Examples 1-4

### Liquid Droplet Formation of Protein Solution Based on Principle of Thermal Ink Jet System

The preparation procedure for each ejection liquid involves dissolving insulin in 0.1 M HCl aqueous solution at an appropriate concentration, then adding an amine represented by the formula (1) (see Table 1) while stirring, and thereafter adjusting the volume with purified water so that desired concentrations of the respective components were obtained.

On the other hand, a liquid ejection head according to the thermal ink jet system having a nozzle diameter of 3 μm was prepared, and a tank connected thereto was filled with a 30% ethanol aqueous solution. The liquid ejection head was driven by a controller electrically connected thereto to eject the liquid from the ejection orifice, and the particle diameter and particle size distribution of the obtained liquid droplets (mist) were measured and confirmed with Spraytec Laser Diffraction Particle Size Analyzer (Malvern Instruments Ltd). As a result, the liquid droplets detected had a sharp particle distribution peak at 3 μm.

The tank connected to the liquid ejection head having the nozzle with a diameter of 3 μm was filled with the ejection liquid prepared by the procedure described above, and the ejection head was driven by the ejection controller to carry out ejection at a frequency of 20 kHz and a voltage of 12 V for 1 second (first ejection). Further, after an interval of 3 seconds, the next 1-second ejection (second ejection) was carried out. This operation was repeated 50 times and the continuity of the ejections was confirmed by visual observation. The ejection continuity (ejectability) was evaluated as ∘ when liquid droplets were ejected 50 times or more; as Δ when the liquid droplet ejection stopped within the range between 15 times to 50 times; and as x when the liquid droplet ejection stopped with operations of less than 15 times. Also, each ejection liquid was subjected to HPLC analyses under predetermined measurement conditions (Equipment: JASCO Corporation; Column: YMC-Pack Diol-200, 500×8.0 mm ID; Eluent: 0.1 M KH₂PO₄—K₂HPO₄ (pH 7.0) containing 0.2M NaCl; Flow rate: 0.7 mL/min; Temperature: 25° C.; Detection: UV at 215 nm) before and after the ejection to confirm the change in the composition of the ejection liquid.

As Comparative Examples, pure water and an insulin solution each not containing the amine compound represented by the formula (1), and ejection liquids containing a substance other than the amine compound represented by the formula (1) were prepared, and the liquid droplet ejection experiments were carried out in the same manner as Examples. The formulations used in the Examples and Comparative Examples and the results are collectively shown in Table 1.

TABLE 1

| | | | | | Surfactant and | | |
|---|---|---|---|---|---|---|---|
| | Protein | | Amine | | additive | | Ejectability |
| | Species | Concentration | Species | Concentration | Type | Concentration | Evaluation |
| Example 1 | Insulin | 4 mg/mL | Ammonia | 50 mg/mL | None | — | ∘ |
| Example 2 | Insulin | 4 mg/mL | Ethylamine | 50 mg/mL | None | — | ∘ |
| Example 3 | Insulin | 4 mg/mL | Trimethylamine | 50 mg/mL | None | — | ∘ |
| Example 4 | Insulin | 4 mg/mL | Hydroxylamine | 50 mg/mL | None | — | ∘ |
| Example 5 | Insulin | 4 mg/mL | Piperidine | 50 mg/mL | None | — | ∘ |
| Example 6 | Insulin | 4 mg/mL | Morpholine | 50 mg/mL | None | — | ∘ |
| Example 7 | Insulin | 4 mg/mL | Ethylenediamine | 25 mg/mL | None | — | ∘ |
| Example 8 | Insulin | 4 mg/mL | Putrescine | 25 mg/mL | None | — | ∘ |
| Example 9 | Insulin | 4 mg/mL | Spermidine | 25 mg/mL | None | — | ∘ |
| Comparative | Water | | None | — | None | — | ∘ |
| Example 1 | | | | | | | |
| Comparative | Insulin | 4 mg/mL | None | — | None | — | x |
| Example 2 | | | | | | | |
| Comparative | Insulin | 4 mg/mL | None | — | Tween 80 | 10 mg/mL | x |
| Example 3 | | | | | | | |
| Comparative | Insulin | 4 mg/mL | None | — | Tween 80 | 50 mg/mL | x |
| Example 4 | | | | | | | |

Note:
Tween 80 is a trade name of polyoxyethylene(20) sorbitan monooleate which is a nonionic surfactant.

Since the pure water of Comparative Example 1 did not contain insulin, the ejections were continued stably. However, in Comparative Examples 2-4 containing insulin, there was no or almost no ejection regardless of the presence/absence of the additive. On the contrary, it can be seen that in Examples 1-9 the ejections were carried out normally and stabilized. The results of the HPLC analyses performed for Examples 1-9 indicated that no change was observed in the peak position and peak area, and in the liquid composition before and after the ejections.

### Examples 10-20 and Comparative Example 5-12

### Effect on Various Proteins and Concentration of Additives

Next, ethylenediamine, putrescine and spermidine, which had stabilized the ejection with a small amount of addition, were selected and added to various proteins at predetermined concentrations. These ejection liquids were evaluated by the same ejection experiments as in Example 1. The formulations investigated in these Examples and the results are collectively shown in Table 2 below.

TABLE 2

| | Protein | | Amines | | Surfactant and additive | | Ejectability |
|---|---|---|---|---|---|---|---|
| | Type | Concentration | Type | Concentration | Type | Concentration | Evaluation |
| Example 10 | Albumin | 1 mg/mL | Ethylenediamine | 10 mg/mL | None | — | ∘ |
| Example 11 | Albumin | 5 mg/mL | Ethylenediamine | 50 mg/mL | None | — | ∘ |
| Example 12 | Albumin | 1 mg/mL | Putrescine | 20 mg/mL | None | — | ∘ |
| Example 13 | Albumin | 1 mg/mL | Spermidine | 20 mg/mL | None | — | ∘ |
| Example 14 | Glucagon | 1 mg/mL | Spermidine | 10 mg/mL | None | — | ∘ |
| Example 15 | GLP-1 | 1 mg/mL | Spermidine | 10 mg/mL | None | — | ∘ |
| Example 16 | hGH | 1 mg/mL | Spermidine | 10 mg/mL | None | — | ∘ |
| Example 17 | EPO | 1 mg/mL | Spermidine | 10 mg/mL | None | — | ∘ |
| Example 18 | IFN α | 1 mg/mL | Spermidine | 10 mg/mL | None | — | ∘ |
| Example 19 | IFN γ | 1 mg/mL | Spermidine | 10 mg/mL | None | — | ∘ |
| Example 20 | Calcitonin | 1 mg/mL | Spermidine | 10 mg/mL | None | — | ∘ |
| Comparative | Albumin | 1 mg/mL | None | — | None | — | x |
| Example 5 | | | | | | | |
| Comparative | Glucagon | 1 mg/mL | None | — | None | — | x |
| Example 6 | | | | | | | |
| Comparative | GLP-1 | 1 mg/mL | None | — | None | — | x |
| Example 7 | | | | | | | |
| Comparative | hGH | 1 mg/mL | None | — | None | — | x |
| Example 8 | | | | | | | |
| Comparative | EPO | 1 mg/mL | None | — | None | — | x |
| Example 9 | | | | | | | |
| Comparative | IFN α | 1 mg/mL | None | — | None | — | x |
| Example 10 | | | | | | | |
| Comparative | IFN γ | 1 mg/mL | None | — | None | — | x |
| Example 11 | | | | | | | |
| Comparative | Calcitonin | 1 mg/mL | None | — | None | — | x |
| Example 12 | | | | | | | |

Although the required addition concentration varied depending on the concentration and species of the protein, the addition of the amines represented by the formula (2) resulted in normal ejection based on the principle of the thermal ink jet system for the respective proteins. Therefore, it was confirmed that the amines represented by the formula (2) exhibit excellent effect for the wide range of proteins. Further, the results of the HPLC analyses performed on Examples 10-20 indicated that no change was observed in the peak position and peak area, and in the liquid composition before and after the ejections.

### Examples 21-24 and Comparative Examples 13 and 14

### Synergistic Effect of Amines Represented by Formula (1) and Surfactant

To a solution in which an amine represented by the formula (1) was added to a protein, a surfactant was further added to prepare an ejection liquid. Ejection liquids thus prepared were evaluated by the same ejection experiments as in Example 1. The formulations investigated in these Examples and the results are collectively shown in Table 3 below.

TABLE 3

| | Protein | | Amines | | Surfactant and additive | | Ejectability |
|---|---|---|---|---|---|---|---|
| | Species | Concentration | Species | Concentration | Species | Concentration | Evaluation |
| Example 21 | Insulin | 4 mg/mL | Ethylenediamine | 1 mg/mL | Tween 80 | 10 mg/mL | ∘ |
| Example 22 | Insulin | 4 mg/mL | Spermidine | 2 mg/mL | Tween 80 | 5 mg/mL | ∘ |
| Example 23 | Albumin | 1 mg/mL | Ethylenediamine | 1 mg/mL | Tween 80 | 10 mg/mL | ∘ |
| Example 24 | Albumin | 1 mg/mL | Spermidine | 2 mg/mL | Tween 80 | 10 mg/mL | ∘ |
| Comparative | Albumin | 1 mg/mL | Ethylenediamine | 1 mg/mL | None | — | Δ |
| Example 13 | | | | | | | |
| Comparative | Albumin | 1 mg/mL | Spermidine | 2 mg/mL | None | — | x |
| Example 14 | | | | | | | |

By adding both the amine represented by the formula (1) and the surfactant (Tween 80), it was possible to normally eject a protein solution with an amine concentration which is far lower than that when the amine is added alone. Further, the ejection was possible even at concentrations at which ejection was not possible when the amine was used alone. The total amount of additives can also be reduced remarkably. Further, by this synergistic effect, it has become possible to eject a protein solution at a higher concentration. Moreover, the results of the HPLC analyses performed on Examples 21-24 indicated that no change was observed in the peak chart and in the liquid composition before and after the ejections.

### Example 25

### Production of Antibody Chip and Sensing Using Ink Jet Printer

Each of Human IL-2 monoclonal antibody, human IL-4 monoclonal antibody and human IL-6 monoclonal antibody was adjusted to concentrations of 0.1 μg/mL to 500 μg/mL. To these solutions, spermidine was added so as to attain a concentration of 1% (w/w) to thereby prepare ejection liquids. Each of the ejection liquids was filled into a head of an ink jet printer (trade name: PIXUS950i; manufactured by Canon Inc.) and respectively ejected on a glass plate coated with Poly-L-Lysin to form spots of each antibody in a predetermined disposition pattern.

FIG. 7 is a model view of the present Example. In FIG. 7, reference numeral 30 denotes a substrate; 31 denotes a masking agent; 32 denotes a substance that specifically reacts with a test substance (protein, peptide, etc.); 33 denotes a test substance; 34 denotes a substance that specifically reacts with the test substance; and 35 denotes a label.

The glass plate, to which the liquid was applied, was incubated at 4° C. and the glass surface was then masked with 1% BSA. After masking, the glass plate was cleaned well to prepare an antibody chip substrate. Next, each of the test substances, recombinant IL2, IL4 and IL6 was used to prepare a solution of a concentration of 1 μg/mL and mixed with spermidine at 1.0% (w/w), a nonionic surfactant (polyoxyethylene(20) sorbitan monolaurate; trade name: Tween 20) at 0.5% (w/w) and BSA at 0.1% (w/w). Each of the liquids was filled into a head of an ink jet printer (trade name: PIXUS950i; manufactured by Canon Inc.) and ejected on the aforementioned antibody chip substrate in the same pattern. The antibody chip substrate, to which the test substance was applied, was covered with a cover glass and a reaction was effected at 4° C. After the reaction, the antibody chip was cleaned well and dried to prepare a detection substrate.

Next, labeling was carried out to detect the test substance captured on the detection substrate. Each of biotin-labeled antibody liquids (biotinylated anti-human IL-2 monoclonal antibody, biotinylated anti-Human IL-4 monoclonal antibody and biotinylated anti-Human IL-6 monoclonal antibody) as substances capable of being specifically bonded to the test substances was dissolved at 1 μg/mL, and spermidine, Tween 20 and BSA were added thereto so as to attain final concentrations of 1.0% (w/w), 0.5% (w/w) and 0.1% (w/W), respectively. Each of the liquids was filled into a head of an ink jet printer (trade name: PIXUS950i; manufactured by Canon Inc.) and ejected on the aforementioned detection substrate in the same pattern. The detection substrate, to which the label was applied, was covered with a cover glass and a reacted was effected at 4° C. After the reaction, the detection substrate was cleaned well and dried.

In order to optically detect the labels, Cy3-labeled streptavidin was dissolved at 10 μg/mL, and spermidine, Tween 20 and BSA were added thereto so as to attain final concentrations of 1.0% (w/w), 0.5% (w/w) and 0.1% (w/w), respectively. Each of the liquids was filled into a head of an ink jet printer (trade name: PIXUS950i; manufactured by Canon Inc.) and ejected on the aforementioned detection substrate in the same pattern. After the ejection operation, the detection substrate was covered with a cover glass and a reaction was effected at 4° C. After the reaction, the detection substrate was cleaned well and dried. Then, the detection substrate was irradiated with an excitation light and the light emission quantity of the Cy3 was measured in terms of the amount of fluorescent signal using a fluorescent scanner equipped with a filter of a transmission wavelength of 532 nm. As a result, there could be detected fluorescent signals which depended on the kinds and concentrations of the sample.

The present invention is not limited to the above embodiments and various changes and modifications can be made within the spirit and scope of the present invention. Therefore to apprise the public of the scope of the present invention, the following claims are made.

This application claims priority from Japanese Patent Application No. 2005-133993 filed on May 2, 2005, which is hereby incorporated by reference herein.

1. An ejection liquid to be ejected from an ejection orifice utilizing a thermal energy for ejection comprising:
at least one of proteins and peptides;
at least one selected from amines represented by the formula (1): 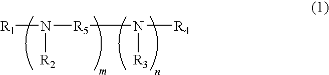
(wherein
R₁ and R₄ are each independently a hydrogen atom, a hydroxyl group, or a substituted or unsubstituted, linear or branched alkyl group having 1 to 8 carbon atoms:
each R₂ and each R₃ is independently a hydrogen atom, a hydroxyl group, or a substituted or unsubstituted, linear or branched alkyl group having 1 to 8 carbon atoms;
adjacent ones of R₁, R₂, R₃ and R₄ may be joined to form a substituted or unsubstituted heterocyclic ring;
each R₅ is independently an alkylene chain having 1 to 8 carbon atoms;
m is an integer of 0 or more; and
n is an integer of 1 or more) and salts thereof; and
a liquid medium comprising water as a main component.

2. The ejection liquid according to claim 1, wherein the amines are ethylenediamine, putrescine, spermidine and derivatives thereof.

3. The ejection liquid according to claim 1 or 2, wherein the at least one of proteins and peptides is at least one of substances selected from calcitonin, insulins, glucagons, interferons, protease inhibitors, cytokines, growth hormones, hematopoietic factors proteins, antibodies, and analogs and derivatives thereof.

4. The ejection liquid according to any one of claims 1 to 3, further comprising a surfactant.

5. The ejection liquid according to claim 4, wherein the surfactant is polyoxyethylene sorbitan fatty acid ester.

6. An ejection method comprising ejecting the ejection liquid set forth in any one of claims 1 to 5 based on a principle of an ink jet system.

7. The ejection method according to claim 6, wherein the ink jet system is a thermal ink jet system.

8. A liquid ejection cartridge comprising a tank for containing the ejection liquid set forth in any one of claims 1 to 5 and an ejection head.

9. The liquid ejection cartridge according to claim 8, wherein the ejection head ejects a liquid by a thermal ink jet system.

10. An ejection apparatus comprising the cartridge set forth in claim 8 or 9, and a flow path and an orifice for leading a liquid ejected from a liquid ejecting portion of a head of the cartridge to an inhalation part of a user.

11. The ejection apparatus according to claim 10, which is for inhalation through a mouth of a user.

12. A method of forming droplets of a liquid comprising at least one of proteins and peptides by applying an energy for ejection to the liquid, which comprises the step of applying an energy for ejection to the liquid filled in a flow path to thereby eject a liquid droplet from an ejection orifice communicating with the flow path, wherein the liquid is the ejection liquid set forth in any one of claims 1 to 5.

13. The method according to claim 12, wherein the liquid droplet is ejected based on a principle of a thermal ink jet system.
